(19) United States
(12) Patent Application Publication
(10) Pub. No.: US 2003/0036565 A1
(43) Pub. Date: Feb. 20, 2003

(54) Cancer chemopreventive agents

(75) Inventors: Kirk L. Parkin, Middleton, WI (US); Mahinda Wettasinghe, Edmonton (CA)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(73) Assignee: WISCONSIN ALUMNI RESEARCH FOUNDATION, MADISON, WI

(21) Appl. No.: 10/178,399

(22) Filed: Jun. 24, 2002

Related U.S. Application Data
(60) Provisional application No. 60/300,509, filed on Jun. 22, 2001.

(51) Int. Cl. A61K 31/12

(52) U.S. Cl. 514/683

(57) ABSTRACT

Extracts prepared from red and high-pigment beetroots (Beta vulgaris L.) with a solvent, such as a water-containing solvent, possess antioxidant activity as demonstrated by a panel of assays. These extracts also have an ability to induce quinone reductase in Murine hepatoma cell (Hepa 1c1c7) cultured in vitro. Fractions purified from the active extracts also possess antioxidant activity and retain quinone reductase-inducing activity in the Hepa 1c1c7 cell line. The active extracts, purified by column, thin layer, and high-performance liquid chromatographic techniques, include betalains. A method of extracting a betalain includes steps of freeze-drying a source containing the betalain; grinding the freeze-dried source; and extracting the betalain from the ground source with the solvent. Additionally, the betalain extract can be isolated betalain components, such as by chromatography.

### CROSS-REFERENCE TO RELATED APPLICATIONS

This application claims priority under 35 U.S.C. §119(e) to U.S. Provisional Patent Application No. 60/300,509, filed Jun. 22, 2001, the entirety of which is incorporated by reference herein.

### REFERENCE TO GOVERNMENT GRANT

This invention was made with United States government support awarded by the USDA, Grant No. 97-36200-5189. The United States has certain rights in this invention.

### BIBLIOGRAPHY

Complete bibliographic citations of the references referred to herein by the first author's last name in parentheses can be found in the Bibliography section, immediately preceding the claims.

### FIELD OF THE INVENTION

The invention relates to cancer chemopreventive agents and antioxidants. In particular, the invention relates to cancer chemopreventive agents and antioxidants derived from sources, such as plants and fungi.

### DESCRIPTION OF THE RELATED ART

Vegetables contain nutritive constituents, such as vitamins and minerals, in abundance and represent a substantial portion of the daily food intake of individuals. They also contain nonnutritive constituents, such as fiber and phenolic compounds, which have been implicated in beneficial health effects in test animals and humans (Burr et al., 1982; Bresnick et al., 1990). In the past, many of the nonnutritive constituents have been ignored because they were considered biologically inert (Wattenberg 1983, 1996). Recent investigations show that there is a profound link between the dietary habits and the incidence of cancer and heart diseases in humans, and the nonnutritive constituents play a major role in preventing the development of these diseases (Willett, 1994). Many of the beneficial health effects of nonnutritive constituents of vegetables have been known to originate from their antioxidant properties (Velioglu et al., 1998).

Oxidative stress and resulting oxidative damage has also been implicated in cancer formation. Many antioxidants are also believed to protect against cancer. Antioxidants reduce or prevent oxidation and have the ability to counteract the damaging effects of free radicals in tissues. Free radicals are highly reactive chemicals that often contain oxygen. Free radicals are produced when molecules are split to form products that have unpaired electrons. Certain antioxidants are also believed to protect against a variety of other diseases including atherosclerosis and heart disease. Antioxidants have been shown to slow the aging process and to slow the progression of Alzheimer's disease. Therapy using antioxidants has the potential to prevent, delay, or ameliorate many neurologic disorders. Thus, the potential health benefits of antioxidants are numerous.

The process of cancer development has three major stages, namely initiation, promotion, and progression (Murakami et al., 1996). The stages of cancer are driven by a variety of mechanisms. For example, cancer can result from tumor suppressor genes being silenced, such as by aberrant methylation. Other types of cancers result from exposure to an initiator and then to a tumor promoter, such as 12-O-tertradecanoylphorbol-13-acetate (TPA).

The initiation stage can be triggered when procarcinogens are converted to carcinogens by phase I enzymes such as cytochromes P-450- and P-448-dependent monooxygenases (Ioannides and Parke, 1987). The carcinogens thus formed are highly electrophilic. They react with cellular macromolecules such as deoxyribonucleic acid (DNA), ribonucleic acids (RNA) and proteins (Counts and Goodman, 1994). These reactions cause mutations in genetic materials, causing cells to proliferate uncontrollably and eventually promote the cancer development.

Mechanisms have been evolved to counteract the harmful activities of phase I enzymes and their reaction products. One such mechanism is the detoxification of activated electrophiles (carcinogens) by phase II enzymes, such as oxidoreductases (e.g., quinone reductase) and transferases (e.g., glutathione transferase). The detoxification process involves the conversion of electrophiles into inactive, more water-soluble and readily excretable conjugates (Talalay, 1989). Phase II enzymes also compete with phase I activating enzymes to limit the generation of electrophiles, thus reducing the risk of initiation. Therefore, it is important to maintain healthy levels of phase II enzymes in bodily tissues in order to fight against highly reactive electrophiles. Several lines of evidence also provide compelling support for the proposition that induction of enzymes of xenobiotic metabolism, and particularly phase II enzymes, results in protection against the toxic and neoplastic effects of carcinogens. For example, various synthetic organic compounds, such as β-naphthoflavone, tert-butylhydroquinone (TBHQ), butylated hydroxytoluene (BHT) and butylated hydroxyanisole (BHA), have been reported to be potent chemopreventive agents because they can induce phase II enzyme synthesis in cultured murine hepatoma cells (Wilkinson and Clapper, 1997). Similarly, many of the nonnutritive food components, such as phenolics (de Long et al., 1986), sulfur-containing compounds (Prochaska et al., 1992; Guyonnet et al., 1999) and glucosinolates and their metabolites (Tawfiq et al. 1995), have also shown chemopreventive properties.

The phase II enzyme inducing activities of crude extracts of vegetables have been documented by Prochaska et al. (1992). Prochaska et al. examined vegetables belonging to families Chenopodiaceae (e.g., beetroots), Compositae (e.g, lettuce), Cruciferae (e.g., broccoli), Cucurbitaceae (e.g., zucchini), Leguminosae (e.g., beans), Liliaceae (e.g., asparagus), Solanaceae (e.g., tomatoes), and Umbelliferae (e.g., carrots). The activities observed for green onion, broccoli, bok choi, and kale were superior amongst 25 vegetables investigated in their study. Prochaska, et al. found that acetonitrile extracts of cruciferous tissues increased the levels of quinone reductase in Hepa 1c1c7 cells. In contrast, extracts of beets using pure acetonitrile were found only minimally inducing of quinone reductase in Hepa 1c1c7 cells.

The beet is a member of the family Chenopodiaceae (goosefoot family) and has been cultivated for over 2000 years. Among its numerous varieties are the garden beet, the sugar beet, beet leaf, i.e., Swiss chard, and several types of mangel-wurzel and other stock feeds. Both the roots and foliage of the red beet are edible, as is the foliage of Swiss chard and similar varieties. The foliage of the sugar beet and several other varieties is used as animal feed. The sugar beet provides about one third of the world's commercial sugar production. In the United States, sugar beets are grown extensively from Michigan to Idaho and in California, accounting for more than half of the United States' sugar production. Beets are classified in the division Magnoliophyta, class Magnoliopsida, order Caryophyllales, and family Chenopodiaceae.

Red beetroots (Beta vulgaris) are an excellent source of red and yellow pigments (Bokern et al., 1991), which provide a natural alternative to synthetic red dyes and have attracted the interest of the natural colorant industry. Beet pigments, collectively known as “betalains,” and beetroot powder have been used as natural colorants in food products such as processed meat, ice cream, baked goods, candies, and yogurt (von Elbe et al., 1974; Vereltzis and Buck, 1984; Vereltzis et al., 1984; Delgado-Vargas et al., 2000). Betalains have been successfully used in commercial food coloring for a number of years, and continue to be an important source of red color in the food industry. There are two distinct types of betalains, namely betacyanins, the red betalains, and betaxanthins, the yellow betalains (Kobayashi et al., 2000). These differ by conjugation of a substituted aromatic nucleus to the 1,7-diazaheptamethinium chromophore, which is present in betacyanin.

Besides imparting attractive color to food products, crude preparations of beet pigments are known to confer free radical scavenging/antioxidant activities (Escribano et al., 1998; Zakharova and Petrova, 1998). The specific source of the antioxidant activities in the crude preparations of red beetroots was not previously identified. It is possible that the antioxidant activities may arise from an array of chemically diverse compounds that include tocopherols, phenolic acids, and their esters, pigments, aromatic peptides, hydrocarbons, and other naturally occurring antioxidants. Antioxidants from various sources have also been implicated in cancer chemoprevention due mainly to their direct involvement in eliminating carcinogens, such as free radicals in humans (Wattenberg, 1996). In addition, it is also believed that some phenolic antioxidants could also play a major role in cancer chemoprevention because they could induce the synthesis of phase II detoxifying enzymes.

Beetroot crude extract has also been found to have an inhibitory effect against TPA-induced promotion of mice skin tumors and against glycerol-induced promotion of lung tumors. (Kapadia, et al. 1996). Kapadia et al. have evidence to show that beetroot crude extract may be useful in inhibiting the second stage of cancer formation. However, Kapadia et al. provided no evidence that beetroot crude extract was useful in preventing cancer initiation, such as by detoxifying the body of pro-carcinogens and carcinogens. Furthermore, Kapadia et al. did not identify the active agent in their beetroot crude extract.

Accordingly, the need exists for methods of increasing the chemoprotective amount of a Phase II enzyme. In addition, there is a need for methods of extracting and isolating betalains from sources of betalains, such as plants and fungi.

### SUMMARY OF THE INVENTION

The inventors have found that extracts prepared from red and high-pigment beetroots (Beta vulgaris L.) with a solvent, such as a water-containing solvent possess antioxidant activity as demonstrated by a panel of assays. These extracts also have an ability to induce quinone reductase in Murine hepatoma cell (Hepa 1c1c7) cultured in vitro. Quinone reductase is a Phase II enzyme, which detoxifies and competes with activated electrophiles (carcinogens). Phase II enzymes also complement phase I activating enzymes to limit the accumulation of electrophiles, thus reducing the risk of initiation. At least two fractions purified from the active extracts are present in the beetroots, and these active fractions also possessed antioxidant activity.

The purified chromatography fractions retained quinone reductase-inducing activity in the Hepa 1c1c7 cell line. The active extracts and fractions thereof that had the chemopreventive action and the antioxidant potential, purified by column, thin layer, and high-performance liquid chromatographic techniques, include various betalains that are identifiable by mass spectroscopy.

A method of producing highly purified beetroot extracts and fractions thereof is provided.

This invention provides for additional value of beetroots and extracts as products of commerce. The isolated compounds can be used as multifunctional ingredients for color, antioxidant, and chemopreventive qualities. These benefits are also applicable to any other betalain-containing material, e.g., Swiss chard and sugar beets. Wastewater from beetroot processing could also serve as a source of betalains, e.g., using spray-drying to concentrate the preparation. Betalains are also present in a variety of other plants and fungi.

### BRIEF DESCRIPTION OF THE DRAWINGS

Preferred exemplary embodiments of the invention are illustrated in the accompanying drawings, in which like reference numerals represent like parts throughout and in which:

FIGS. 1A and 1B are graphs showing the total antioxidant activity of aqueous (1A) and ethanolic (1B) extracts of beetroots. Bars sharing the same letter in a single bar chart are not significantly different (p>0.05) from one another.

FIGS. 2A and 2B are graphs illustrating the total reducing power of aqueous (2A) and ethanolic (2B) extracts of beetroots. Bars sharing the same letter in a single bar chart are not significantly different (p>0.05) from one another.

FIGS. 3A and 3B are graphs demonstrating the oxygen radical absorbance capacity (ORAC) of aqueous (3A) and ethanolic (3B) extracts of beetroots. Bars sharing the same letter in a single bar chart are not significantly different (p>0.05) from one another.

FIGS. 4A to 4D are graphs showing the effect of aqueous (4A, 4C) and ethanolic (4B, 4D) extracts of red beets on induction of quinone reductase specific activity and cell density. Legends followed by the same letter in a single graph indicate that there are no significant differences (p>0.05) among the highest ratio quinone reductase specific activity or the lowest cell density observed in the varieties tested.

FIG. 5 is a graph depicting the dependence of ratio quinone reductase specific activity on the concentration of β-naphthoflavone.

FIGS. 6A to 6D are column chromatographic fraction profiles of aqueous extracts of red (6A) and high-pigment (6B) beetroots, and ethanolic extracts of red (6C) and high-pigment (6D) beetroots.

FIGS. 7A to 7D are graphs showing total antioxidant activity of column fractions of aqueous and ethanolic extracts of beetroots. (7A), aqueous red; (7B), ethanolic red; (7C), aqueous high-pigment; (7D), ethanolic high-pigment. Bars sharing the same letter in a single bar chart are not significantly different (p>0.05) from one another.

FIGS. 8A to 8D are graphs illustrating the total reducing power of column fractions of aqueous and ethanolic extracts of beetroots. (8A), aqueous red; (8B), ethanolic red; (8C), aqueous high-pigment; (8D), ethanolic high-pigment. Bars sharing the same letter in a single bar chart are not significantly different (p>0.05) from one another.

FIGS. 9A to 9D are graphs showing the oxygen radical absorbance capacity (ORAC) of column fractions of aqueous and ethanolic extracts of beetroots. (9A), aqueous red; (9B), ethanolic red; (9C), aqueous high-pigment; (9D), ethanolic high-pigment. Bars sharing the same letter in a single bar chart are not significantly different (p>0.05) from one another.

FIGS. 10A to 10D are graphs depicting the effect of column fractions of aqueous (10A, 10C) and ethanolic (10B, 10D) extracts of red beetroots on induction of quinone reductase specific activity and cell density. Legends followed by the same letter in a single graph indicate that there are no significant differences (p>0.05) among the highest ratio QR specific activity or the lowest cell density observed in the varieties tested.

FIGS. 11A to 11D are graphs demonstrating the effect of fractions of aqueous (11A, 11C) and ethanolic (11B, 11D) extracts of high-pigment beetroots on induction of quinone reductase specific activity and cell density. Legends followed by the same letter in a single graph indicate that there are no significant differences (p>0.05) among the highest ratio QR specific activity or the lowest cell density observed in the varieties tested.

Before explaining embodiments of the invention in detail, it is to be understood that the invention is not limited in its application to the details of construction and the arrangement of the components set forth in the following description or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION

Abbreviations:

The following abbreviations are used herein: DNA, deoxyribonucleic acid; RNA, ribonucleic acid; TBHQ, tert-butylhydroquinone; BHT, butylated hydroxytoluene; BHA, butylated hydroxyanisole; ABTS, 2,2′-azinobis(3-ethylbenzthiazoline-6-sulfonic acid; AAPH, 2,2′-azobis-(2-amidinopropane) dihydrochloride; SDS, sodium dodecylsulphate; MTT, 3-(4,5-dimethylthiazo-2-yl)-2,5-diphenyltetrazolium bromide; FAD, flavin adenine dinucleotide; NADP, nicotinamide adenine dinucleotide phosphate; MEM, minimum essential medium; QR, quinone reductase.

Definitions:

The following definitions are intended to assist in providing a clear and consistent understanding of the scope and detail of the terms:

Betalains: derivatives of betalamic acid, which can be classified into two structural groups: red betacyanins and yellow betaxanthins.

Chemoprotector or chemoprotectant: a synthetic or naturally occurring chemical agent that reduces susceptibility in a mammal to the toxic and neoplastic effects of carcinogens.

Inducer activity or Phase II enzyme-inducing activity: a measure of the ability of a compound(s) to induce Phase II enzyme activity. In the present invention, inducer activity is measured by the murine hepatoma cell bioassay of QR activity in vitro. Inducer activity is defined herein as QR inducing activity in Hepa 1c1c7 cells (murine hepatoma cells) incubated with extracts of beetroots.

In a preferred embodiment, betalain extracts are recovered from beetroots as is described below. The extracts have antioxidant properties. The extracts also increase levels of quinone reductase in murine hepatoma cells. Quinone reductase is a Phase II enzyme that has been shown to protect against the toxic and neoplastic effects of carcinogens. The extracts preferably are prepared in a water-containing solvent, such as hot water or aqueous (95%) ethanol, following freeze-drying. Further purification of the extracts by column chromatography produces several fractions, some of which retain the quinone reductase inducer activity and antioxidant property. The active fractions of the extracts contain betalains.

Method of Preparing and Purifying Extracts.

The present invention includes novel techniques to purify extracts having Phase II enzyme inducing activities and antioxidant properties from beetroots, other plants, fungi, and other sources of betalains. A preferred method of preparing extracts from beetroots and other sources of betalains includes freeze-drying and then extracting the resulting freeze-dried powder with either hot water or aqueous (95%) ethanol. Freeze-drying preferably includes steps of dicing, freezing, and lyophilizing peeled beetroots. The lyophilized beets are then ground into a fine powder, which can be stored until used. Preferably, particles of the fine powder have a diameter of about 420 microns or less. More preferably, the particles have a diameter in the range of about 297 microns to about 420 microns

The freeze-dried powder then is extracted preferably by a solvent, such as a water-containing solvent, for example, water, aqueous ethanol (preferably 95% ethanol and 5% water (v/v)), 90% methanol, 80% ethanol, and 80% acetonitrile. For aqueous, i.e., pure water, extracts, boiling water is added to the powdered beetroots. The mixture is blended and filtered, such as through cheesecloth. The filtrate is then centrifuged. The supernatant is collected, frozen, and lyophilized. The lyophilized extract can be stored until further use. For aqueous ethanol extracts, aqueous ethanol, (preferably 5 and 95%, v/v, water and ethanol, respectively, and preferably heated to 60° C.) is added to the powdered beetroots. The resulting slurry is filtered and then evaporated to remove the ethanol. The concentrated extract can be frozen, lyophilized, and stored until further use.

Chromatography can be used to isolate the extracts into components. In a preferred chromatography method, the lyophilized crude extract is dissolved in a solvent (preferably 50% (v/v) methanol for the aqueous extracts or 95% (v/v) methanol for the aqueous ethanol extracts). Liquid extracts are then applied to a column packed with Sephadex LH-20 (particle size 25-100 μm) and eluted with either 50% (v/v) methanol or 95% (v/v) methanol. Fractions of eluate are collected via a fraction collector. The absorbance of the fractions is then measured at 280 nm. Fraction profiles are constructed, and the major fractions identified and collected by pooling the contents of appropriate collection tubes. The methanol is evaporated. The resulting concentrated fractions are frozen and lyophilized. The lyophilized fractions can be stored until used.

Antioxidant Potential of the Beetroot Extracts and Fractions Thereof.

The antioxidant potential of the extracts and the fractions thereof generated from red beets and high-pigment beets is demonstrated by several assays. First, the extracts and fractions thereof hinder the generation of ABTS free radicals. Both aqueous and aqueous ethanolic extracts of both red and high-pigment beets show significant total antioxidant activities. The column chromatography purified fractions of the extracts show strong inhibition of ABTS free radical generation, demonstrating the total antioxidant activity of the fractions.

Second, the red beet and high-pigment beet extracts and fractions thereof also possess the ability to reduce pre-formed ABTS free radicals into their neutral form through electron and/or hydrogen atom donation. Aqueous and ethanolic extracts of all beetroot varieties, at all concentrations tested, significantly (p<0.05) highly reduce ABTS free radicals as compared to controls of Trolox® (6-hydroxy-2,5,7,8-tetramethylchroman-2-carboxylic acid), which is a cell-permeable, water-soluble derivative of vitamin E with potent antioxidant properties. Several fractions of aqueous red beetroot extracts completely (100%) reduce ABTS radical generation under the conditions assayed. A fraction of the ethanolic red beetroot extracts also showed ˜100% reduction of the preformed ABTS radical, whereas the remaining fractions showed <50% reduction. For the high-pigment beetroots, many aqueous and ethanolic extracts showed a complete (100%) reduction of generation of the ABTS free radical under the conditions assayed. Thus, the beetroot extracts and fractions thereof further demonstrate their antioxidant potential.

Third, ORAC, which measures the ability of the sample being tested to protect against attack by free radicals, or to act as an antioxidant, demonstrates that several extracts protect against attack by free radicals. For example, aqueous extracts of red beetroots and of high-pigment beetroots show significantly (p<0.05) high ORAC as compared to control. One extract of orange beetroots showed a significant difference from that of the control. Ethanolic extracts of red beets and those of high-pigment beetroots exhibited significantly high ORAC as compared to control. Several fractions generated from the extracts also protected against attack by free radicals as demonstrated by ORAC.

Thus, the beetroot extracts and fractions thereof are capable of acting as antioxidants. Uses of antioxidants include, but are not limited to, the treatment and prevention of cancer and endothelial injury, such as ischemic and reperfused myocardium. Because of their antioxidant activity, the extracts and fractions thereof of this invention may also be used in treating and preventing cancerous conditions by, for example, preventing cancer-causing mutations in the genetic material of an animal or a human.

Because of the antioxidant activity of beetroot extracts and fractions thereof, antiatherogenic diseases and conditions that may be treated using beetroot extracts and fractions thereof include, but are not limited to, arteriosclerosis, atherosclerosis, myocardial infarction, ischemia (e.g., myocardial ischemica, brain ischemia and renal ischemia) and strokes.

Phase II Enzyme Inducer Properties of Beetroot Extracts and Fractions Thereof.

Beetroot highly purified extracts and purified fractions thereof also increase the levels of a Phase II enzyme, quinone reductase, as demonstrated in an in vitro assay. The beetroot extracts and purified fractions thereof are important because they have the potential to protect against carcinogens and other electrophiles. They also have the potential to neutralize the effects of free radicals and to recycle antioxidants.

Most of the phase II enzyme inducing properties of red beetroots may be attributable to the constituents or their metabolites whose structure possesses at least one Michael accepter group, which is an olefin conjugated with an electron-withdrawing group. Compounds, whose structure has Michael acceptors, induce the genes responsible for phase II enzyme synthesis through an electrophilic signal as in the case of monofunctional inducers and by binding with aryl hydrocarbon receptor as in the case of bifunctional inducers (Talalay, 1989).

Further Purification and Analysis.

The fractions purified from the red and high-pigment beets and subjected to mass spectroscopy show the presence of several betalains and the betalain precursor, betalamic acid. The identified betalains include vulgaxanthin I and II, betanidin, phyllocactin, 2-descarboxybetanin, betanin, and 5″-O-E-feruloyl-2′-apiosyl-betanin. The fractions also contain several other unidentified betalains.

The highly purified fractions from the beetroots, which almost exclusively contain betalains, possess antioxidant activity and induce Phase II enzymes. Thus, the betalains confer antioxidant properties and induce Phase II enzymes.

The invention therefore provides a new source of a Phase II enzyme inducer. Food products therefore can be prepared to include higher levels of betalains by adding the extracts described herein and the fractions described herein to food products. For example, purified betalain-containing extracts and fractions thereof can be concentrated and dried to form powders that can be added to food products or from which food products can be made. Powders can be made into pills, such as gel capsules containing the powders or compressed tablets that are formed by adding a binding agent. The betalain-containing extracts can also be consumed as liquids, such as tinctures or teas.

Additional Sources of Betalains.

Betalain extracts and betalains themselves may be recovered from any biological materials including, but not limited to, any and all plants of the following taxonomic families: Amaranthaceae, Aizoaceae, Basellaceae, Chenopodiaceae, Cactaceae, Nyctaginaceae, Phytolaccaceae, Portulacaceae, and Didieraceae. Betalain extracts and betalains themselves may also be recovered from higher fungi such as Amanita, Hygrocybe, and Hygrosporus. Examples of plants containing betalains and some of the tissues from which they can be extracted include, but are not limited to, Alzoaceae flowers, Cactaceae fruits, including Christimas cactus flower petals (Schlumbergera×buckleyi), Phyllocactus hybridus, and Prickly pear (Opuntia ficus-indica), Bougainvillea (Nyctagynaceae) bracts, Teloxis, leaf beet, i.e., Swiss chard (Beta vulgaris var. cicla), Basella fruits (Basella rubra L.), Amaranthus seeds (Amaranthus tricolor), Gomphrena globosa, Mirabilis (Mirabilis jalapa) flowers, Portulaca grandiflora flowers, Chenopodiun rubrum, Lampranthus, sociorum, Riviana humilis, Carpobrotus acinaciformis, Agaricales (Amantia muscaria) mushroom, Iresine lindenii, and Phytolacca americana.

Common names and classification of different betacyanins and betaxanthins are standardized, and they are usually assigned in agreement with their botanical genus from which the betalain was originally isolated. In the betacyanin group, amaranthin-I was obtained from Amaranthus tricolor, betanin from Beta vulgaris, and gomphrenin-I from Gomphrena globosa. While in the betaxanthin group, miraxanthin occurs in flowers of Mirabilis jalapa, vulgaxanthin-I and II have been found in root of B. vulgaris and portulaxanthin has been isolated from the petals of Portulaca grandiflora. Phyllocactin has been isolated from Phyllocactus hybridus. More than 50 betalains are well known, and all of them have the same basic structure, which is shown below, and in which R₁ and R₂ may be hydrogen or an aromatic substituent.

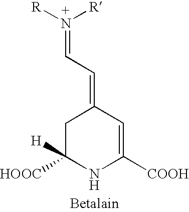

Betacyanins and betaxanthins can be classified by using their chemical structures. Betacyanin structures show variations in their sugar (e.g., 5-O-D-Glucose) and acyl groups (e.g., feruloyl), whereas betaxanthins show conjugation with a wide range of amines and amino acids (e.g., dihydroxyphenylanine, tyrosine, and glutamine) in their structures.

Breeding programs exist for producing high-pigment beets. For example, in 1996, such a breeding program conducted a study to select for beets having an increased betalain pigment concentration but not an increase in total dissolved solids. In this study, a total pigment concentration increased 200% after eight cycles of recurrent selection in two red beet populations selected for high total betalain concentration (Goldman, et al., 1996).

A triallelic system at the R locus with incomplete dominance has been found to control the qualitative expression of the betacyanin:betaxanthine pigment ratio in red table beet. Distinct total pigment concentrations were associated with the R locus genotypes (Wolyn & Gabelman, 1989). Thus, beets can be studied and bred to generate beets containing increased amounts of betalains.

Cell Tissue Culture.

Another source of betalains is cell tissue culture. Cell tissue culture has been a very useful tool in the study of various aspects of biochemistry, enzymology, genetics, and biosynthesis of betalains. Betalain production by plant cell culture represents an excellent option as it has a number of advantages over conventional procedures. For instance with this methodology, it is possible to control quality and availability of pigments independently of environmental changes.

Nutritional Supplements.

Nutritional supplements can be produced from betalain extracts and from fractions purified from the betalain extracts. In a preferred embodiment, a nutritional supplement is produced by spray drying a betalain extract. In another preferred embodiment, a nutritional supplement is produced by spray drying a fraction purified from a betalain extract. For example, extracts purified from high-pigment beets or from red beets are spray dried. The dried extract is then formed into a nutritional supplement by, e.g., adding the dried betalain to a capsule or by forming a pill from the dried betalain. Carriers, fillers, and other ingredients can be added to the dried betalain. In a preferred embodiment, the dietary supplement contains at least 0.3% of a betalain. More preferably, the dietary supplement contains at least about 0.4% and even more preferably, at least about 0.8% of the betalain.

### EXAMPLES

The following Examples are provided for illustrative purposes only. The Examples are included herein solely to aid in a more complete understanding of the presently described invention. The Examples do not limit the scope of the invention described or claimed herein in any fashion.

### EXAMPLE 1

### Samples and Extractions

### Example 1a

Beet Samples.

Red, white, orange and high-pigment beetroot varieties used were obtained from breeding material derived from crosses of inbred lines released by the University of Wisconsin Table Beet Breeding program (Goldman, I. L., 1996).

### Example 1b

Preparation of Beetroot Powder.

Peeled beetroots (400 g) were diced into approximately 1 cm³ cubes, frozen, and lyophilized (Virtis UNI-TRAP, Model 10-100, The Virtis Company, Gardiner, NY) for 72 h at 100 microns. Lyophilized beets were ground (Krups Type 203 household grinder) into a fine powder and stored in glass bottles at 4° C. until used.

### Example 1c

Preparation of Aqueous Extracts.

Powdered beetroots (25 g) were blended with 250 mL of boiling water for 2 min and filtered through a double layer of cheesecloth and then the filtrate was centrifuged at 3180 rpm for 10 min. Supernatant was collected, frozen, and lyophilized for 48 h at 100 microns. Lyophilized extracts were stored in glass bottles at 4° C. until used.

### Example 1d

Preparation of Aqueous Ethanolic Extracts.

One hundred milliliters of aqueous ethanol (5 and 95%, v/v, water and ethanol, respectively) were added into a round-bottom flask containing 6 g of powdered beetroots and heated to 60° C. under reflux for 25 min while stirring in a water bath set at 60° C. The resulting slurry was filtered through a Whatman No. 3 filter paper, and the filtrate was subjected to rotary evaporation (Buchi Rotavapor R110, Flawil, Switzerland) at 40° C. to remove ethanol. The concentrated extracts were frozen and lyophilized for 48 h at 100 microns. Lyophilized extracts were stored in glass vials at 4° C. until used.

### EXAMPLE 2

### Column Chromatography of Extracts

### Example 2a

Sephadex LH-20 Column Chromatography of Extracts.

Five hundred milligrams of extracts were dissolved in 3 mL of 50% (v/v) aqueous methanol for aqueous extracts or 3 mL of 95% (v/v) aqueous methanol for aqueous ethanol extracts. Liquid extracts were then applied to a column (2.5 cm diameter and 75 cm long) packed with Sephadex LH-20 (particle size 25-100 μm) and eluted with either 50% (v/v) methanol or 95% (v/v) methanol. Eluting materials (4 mL) were collected in test tubes placed in a fraction collector (Foxy Jr., ISCO, Inc., Superior St., Lincoln, NE) and their absorbance measured at 280 nm. Fraction profiles were constructed, and the major fractions identified. The contents of the tubes were then pooled into fractions I-IV for aqueous extracts and I-V for aqueous ethanol extracts. Methanol was evaporated under vacuum at 40° C., and the resulting concentrated fractions were frozen and lyophilized for 48 h at 100 microns. Lyophilized fractions were stored in glass bottles at 4° C. until used.

### Example 2b

Sephadex LH-20 Column Chromatography Fractions of Aqueous and Aqueous Ethanol Extracts

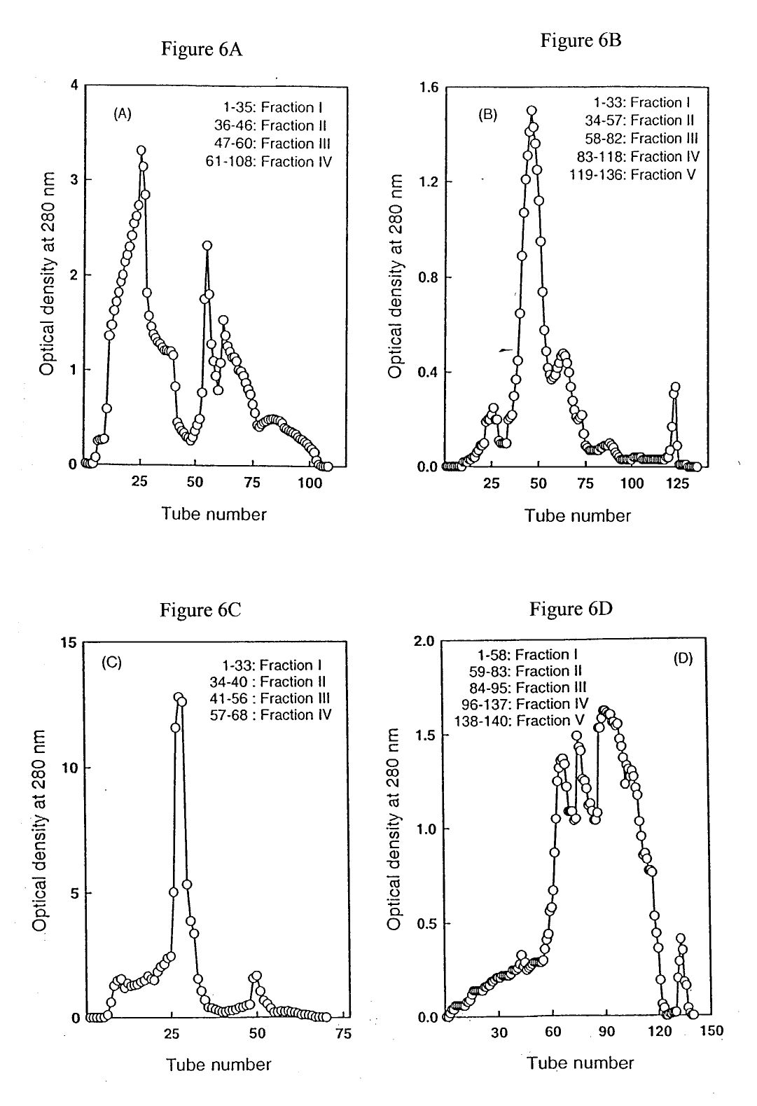

Aqueous extracts of both red and high-pigment beetroots yielded four fractions each when subjected to Sephadex LH-20 column chromatography (FIGS. 6A and C) whereas the ethanolic extracts yielded five fractions each (FIGS. 6B and D). However, the fraction V of ethanolic extracts of high-pigment beetroots was added to fraction IV due to its low (trace) yield of dry matter. Among the fractions of all four types of beetroot extracts, fraction II had the highest yield (˜70-90%, w/w of total yield of dry matter) while the individual yield of remaining fractions was ˜2-14% (w/w of total yield of dry matter). It is noteworthy that there were distinct differences among the fraction colors. Fractions I-IV of aqueous extracts of red and high-pigment beetroots were red, orange, brown and deep purple color, respectively. Fractions I-V of ethanolic extracts of red beetroots were red, yellow, brown, pink, and deep purple color, respectively, while the color of fractions I-III of ethanolic extracts of high-pigment beets was the same as those of aqueous extracts. Fraction IV of ethanolic extracts of high-pigment beetroots was deep purple in color. These fractions were tested for their antioxidant properties and their ability to induce Phase II enzymes, as described below.

### EXAMPLE 3

### Antioxidant Activity

### Example 3a

Total Antioxidant Activity Assay.

All reagents used in this assay were prepared in phosphate buffered saline (PBS, 10 mM, pH 7.4). Sample solutions were prepared in either PBS or 95% ethanol. ABTS (2.5 mM, 100 μL), metmyoglobin (50 μM, 180 μL), PBS (790 μL) and test solution (10 μL; 42, 84 and 210 μg/mL assay medium) were mixed in a disposable cuvette. Reaction was triggered by the addition of hydrogen peroxide (10 mM, 120 μL) into the cuvette. Absorbance data of the reaction mixture were recorded up to 10 min using a spectrophotometer (Beckman DU-65, Beckman Coulter, Inc., Fullerton, CA) set at 734 nm (Rice-Evans and Miller, 1994). Data were plotted against a time scale and the area under curves, calculated using Jandel Scientific software Sigmaplot (San Rafael, CA), was used to calculate % inhibition as follows:

% Inhibition=[(Area_{control}−Area_{treated})/Area_{control}]*100

### Example 3b

Total Antioxidant Activity of Extracts.

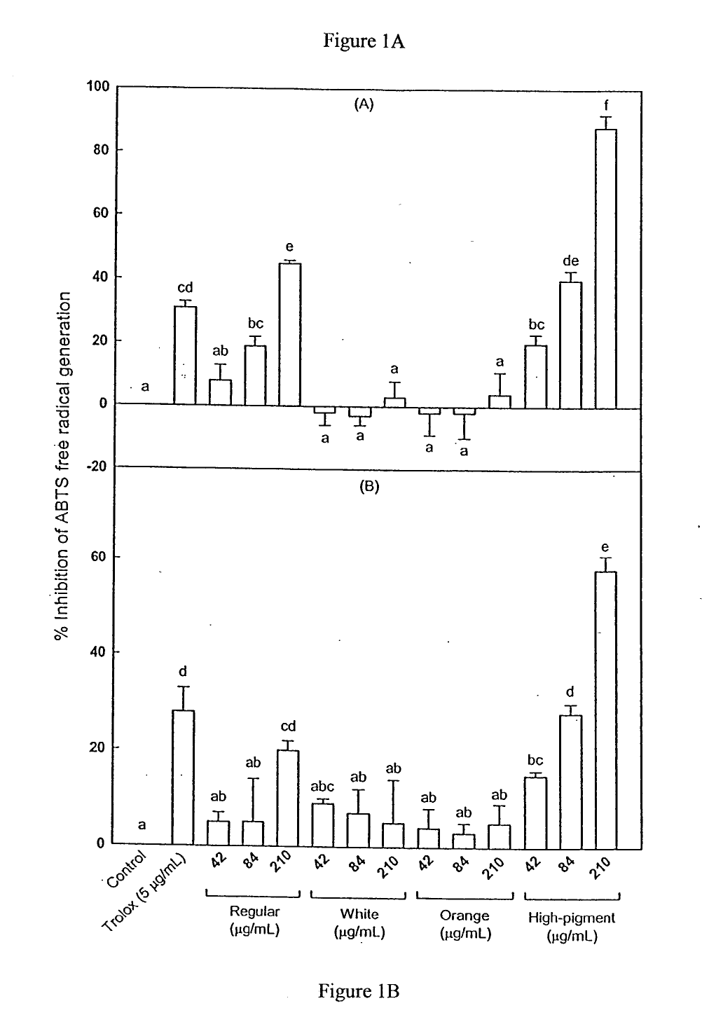

Total antioxidant activity assay measures the ability of test compounds to hinder the generation of ABTS free radicals. FIG. 1A shows the total antioxidant activity of aqueous extracts of beetroot varieties at three different concentrations. The total antioxidant activity of aqueous extracts of red beetroot at 42 μg/mL was not significantly different p>0.05) from that of the control while the 84 and 210 μg/mL concentrations showed a significant (p<0.05) antioxidant activity. The total antioxidant activity of aqueous extracts of red beetroots at 210 μg/mL concentration was better than that evident for Trolox® at 5 μg/mL. All concentrations of aqueous extracts of white and orange beetroots were ineffective (p>0.05). Amongst the aqueous extracts, the greatest total antioxidant activities were seen for the high-pigment beetroot extracts. At 210 μg/mL concentration, the total antioxidant activity of aqueous extracts of high-pigment beetroots was greater (p<0.05) than that exerted by Trolox® at 5 μg/mL.

FIG. 1B shows the total antioxidant activity of ethanolic extracts of beetroots. A significantly (p<0.05) high total antioxidant activity was evident for ethanolic extracts of red beetroots at 210 μg/mL concentration and those of high-pigment beetroots at all concentrations examined. However, the total antioxidant activities observed for ethanolic extracts of red and high-pigment beetroots were inferior to those observed for aqueous extracts at the same concentration (FIGS. 1A and B). Similar to their aqueous counterparts, ethanolic extracts of white and orange beetroots had no significant antioxidant effects (FIG. 1B). These data suggested that the degree and nature of pigmentation had profound effects on total antioxidant potential of beetroots.

### Example 3c

Total Antioxidant Activity of Fractions.

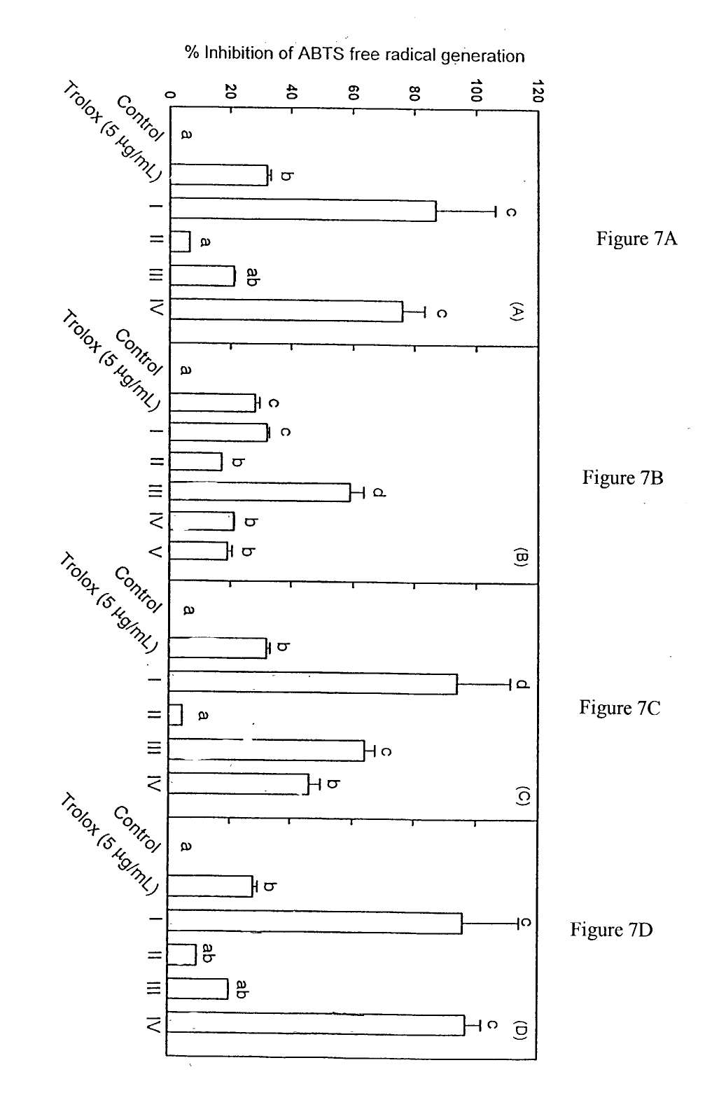

Amongst the column fractions of aqueous red beetroot extracts, fractions I and IV showed strong inhibition of ABTS free radical generation (FIG. 7A), while fractions II and III failed to show a significant (p >0.05) inhibition. Except for the fraction III, which showed ˜60% inhibition, all other fractions of ethanolic red beetroot extracts showed inhibitions <40% (FIG. 7B). Similar to fraction I of aqueous red beetroot extracts, fraction I of aqueous high-pigment beetroot extracts also showed a strong antioxidant activity (˜90% inhibition; FIG. 7C). Fractions III and IV of aqueous high-pigment beetroot extracts exerted ˜40 and ˜60% inhibitions, respectively. Amongst the column fractions of ethanolic high-pigment beetroot extracts, fractions I and IV showed ˜100% inhibition of the ABTS radical generation (FIG. 7D). The other two fractions did not show antioxidant properties. Thus, several fractions of the aqueous and aqueous ethanol extracts retained their antioxidant properties, while other fractions did not.

### EXAMPLE 4

### Reducing Power

### Example 4a

Total Reducing Power Assay.

ABTS radical was generated by mixing five milliliters of an aqueous ABTS solution (7 mM) and 88 μL of a potassium persulfate solution (140 mM), followed by standing in the dark for 6 h. This stock solution (1.1 mL) was diluted to 90 mL with PBS (pH 7.0) and the absorbance of the diluted solution was adjusted to 0.83. Test solution (10 μL; 42, 84 and 210 μg/mL assay medium) and PBS (pH 7.0, 190 μL) were transferred into a disposable cuvette and the assay was started by the addition of 1 mL of the preformed ABTS radical. Absorbance at 734 mL was measured after 10-min (Pellegrini et al., 1999). Percentage reduction was calculated using the following equation:

% Reduction=[(Absorbanceᵢₙᵢₜᵢₐₗ−Absorbance_{final})/Absorbanceᵢₙᵢₜᵢₐₗ]*100

### Example 4b

Total Reducing Power of Extracts.

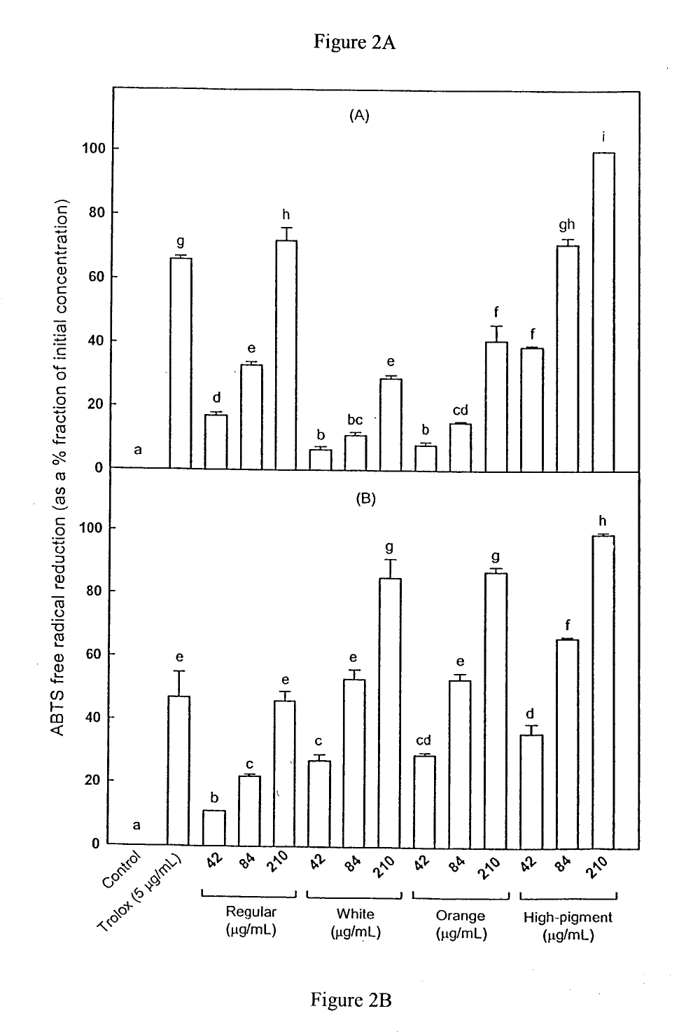

The ability of extracts to reduce pre-formed ABTS free radicals into their neutral form through electron and/or hydrogen atom donation was employed to further assess the antioxidant potential of the extracts. As shown in FIG. 2, aqueous and ethanolic extracts of all beetroot varieties, at all concentrations tested, resulted in a significantly (p<0.05) high reduction of ABTS free radicals as compared to controls. About 70% reduction of ABTS free radicals was evident for 210 μg/mL concentration of aqueous extracts of red beetroots, whereas the reduction brought about by 84 and 210 μg/mL concentration of aqueous extracts of high-pigment beetroots was ˜70% and 100%, respectively (FIG. 2A). For all concentrations tested, the reducing power of ethanolic extracts of red beetroots was inferior to that observed for aqueous extracts (FIG. 2B). Ethanol extracts of white and orange beetroots showed much stronger reducing power than those exerted by their aqueous counterparts at the same concentrations. The reducing power of ethanolic extracts of high-pigment beetroots was similar to those observed for their aqueous counterparts (FIG. 2B). Thus, various aqueous and ethanol extracts of red, high-pigment, white, and orange beetroots had reducing power.

### Example 4c

Total Reducing Power of Fractions.

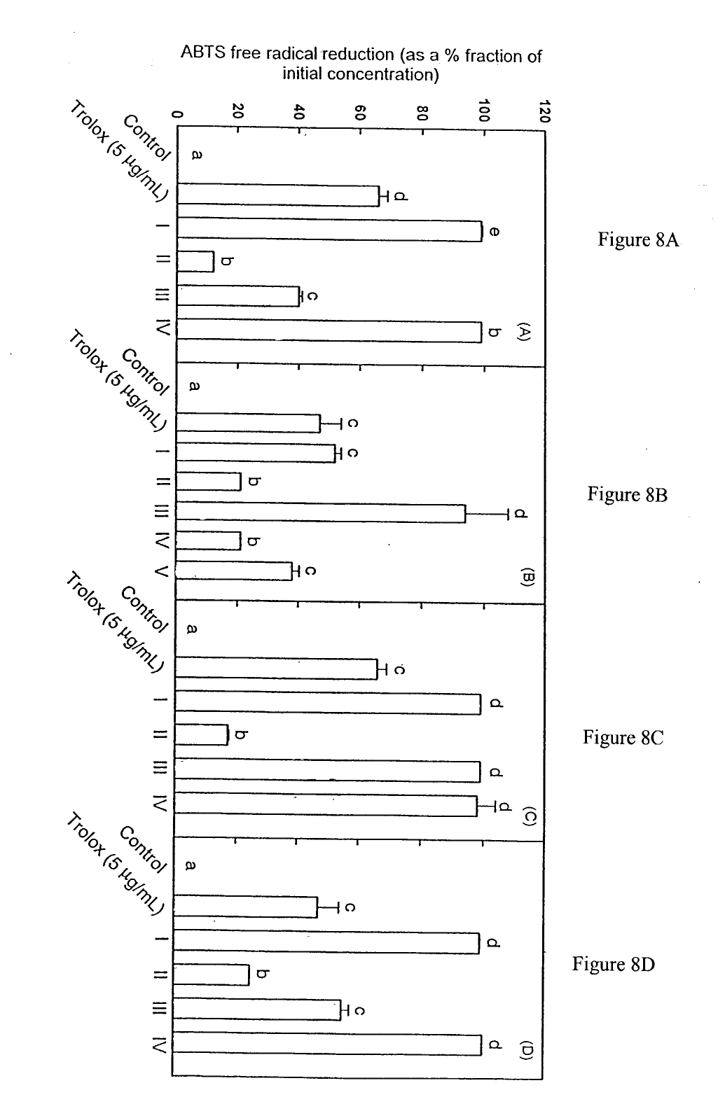

As shown in FIG. 8A, fractions I and IV of aqueous red beetroot extracts completely (100%) reduced the ABTS radical while the other two fractions showed relatively weak reducing properties. Fraction III of ethanolic red beetroot extracts also showed ˜100% reduction of the preformed ABTS radical whereas the remaining fractions showed <50% reduction (FIG. 8B). With the exception of fraction II, which showed weak reducing power, fractions of aqueous high-pigment beetroot extracts showed a complete (100%) reduction of the ABTS free radical (FIG. 8C). Fractions I and IV of ethanolic extracts of high-pigment beetroots exerted strong reducing properties while fraction III possessed moderate reducing power (FIG. 8D). Fraction II was the weakest amongst the four fractions. The reducing power of the various fractions varied, showing that the reducing power was located in certain fractions, thereby further identifying which fractions were active.

### EXAMPLE 5

### OXYGEN RADICAL ABSORBANCE CAPACITY

### Example 5a

Oxygen Radical Absorbance Capacity (ORAC) Assay.

A 0.5M solution of AAPH was prepared in degassed double distilled water and a β-carotene solution was prepared by centrifuging 10 mg of β-carotene in 10 mL of acetone followed by a one-fold dilution of the supernatant with the same solvent. β-Carotene (60 μL) and 0.6% (w/v) phosphate buffered Tween 20 containing 0.3% (w/v) linoleic acid (120 μL) were mixed with 935 μL of PBS in a quartz cuvette and incubated for 2 min at 50° C. Test solution (10 μL; 42, 84 and 210 μg/mL assay medium) and AAPH (25 μL) were added into the cuvette and the reaction was monitored using a spectrophotometer set at 452 nm and 50° C. (Velioglu et al., 1998). Percentage β-carotene retention was calculated using the following equation:

% β-carotene retention=100−[(Absorbanceᵢₙᵢₜᵢₐₗ−Absorbance_{5 min})/Absorbanceᵢₙᵢₜᵢₐₗ]*100

AAPH, used in this assay, is a strong catalyst, which catalyses oxygen free radical generation. The presence of an antioxidant in the assay medium would render these oxygen free radicals into neutral species. This, in turn, would minimize the bleaching of β-carotene, the indicator compound.

### Example 5b

ORAC of Extracts.

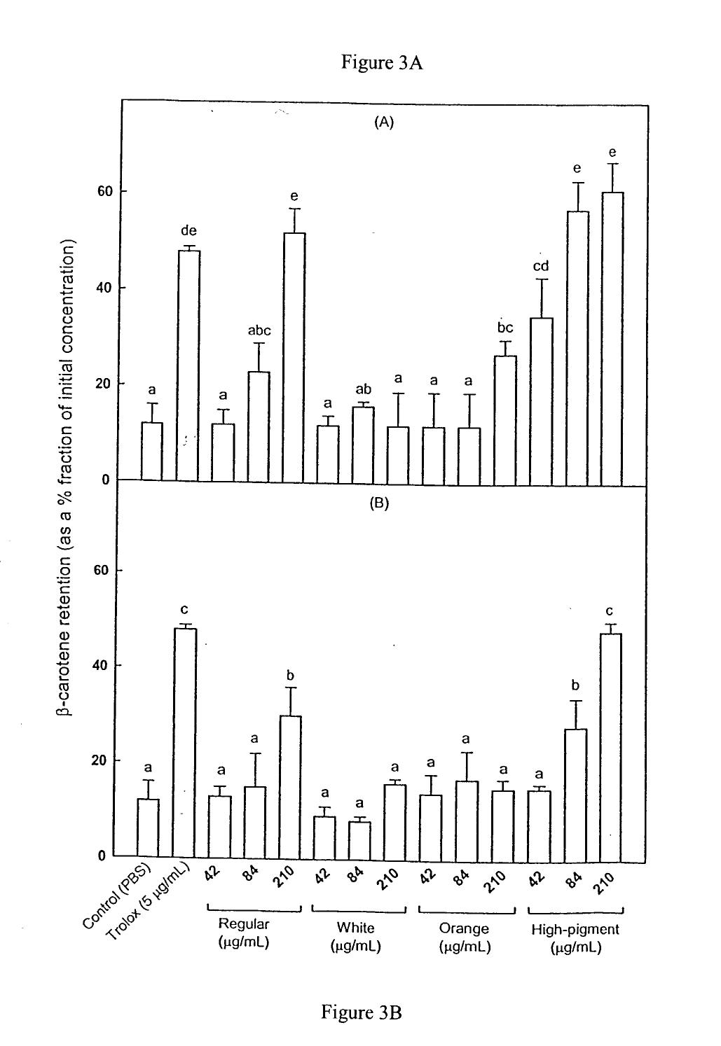

Aqueous extracts of red beetroots at 210 μg/mL concentration and all concentrations of high-pigment beetroots had significantly (p<0.05) higher ORAC as compared to control (FIG. 3A). Except for the 210 μg/mL concentration of orange beetroots, ORAC of aqueous extracts of white and orange beetroots showed no significant (p>0.05) differences from that of the control (FIG. 3B).

Ethanolic extracts of red beets at 210 μg/mL and those of high-pigment beetroots at 84 and 210 μg/mL exhibited significantly higher ORAC as compared to control (FIG. 3B), but the capacities were about 10-20% lower than those observed for aqueous extracts at the same concentration. The ORAC of ethanolic extracts of white and orange beetroots were not different (p>0.05) from that of the control. The ORAC of red and highly-pigmented beetroot extracts further demonstrates that they have ability to protect against attack by free radicals and to act as an antioxidant.

### Example 5c

ORAC of Fractions.

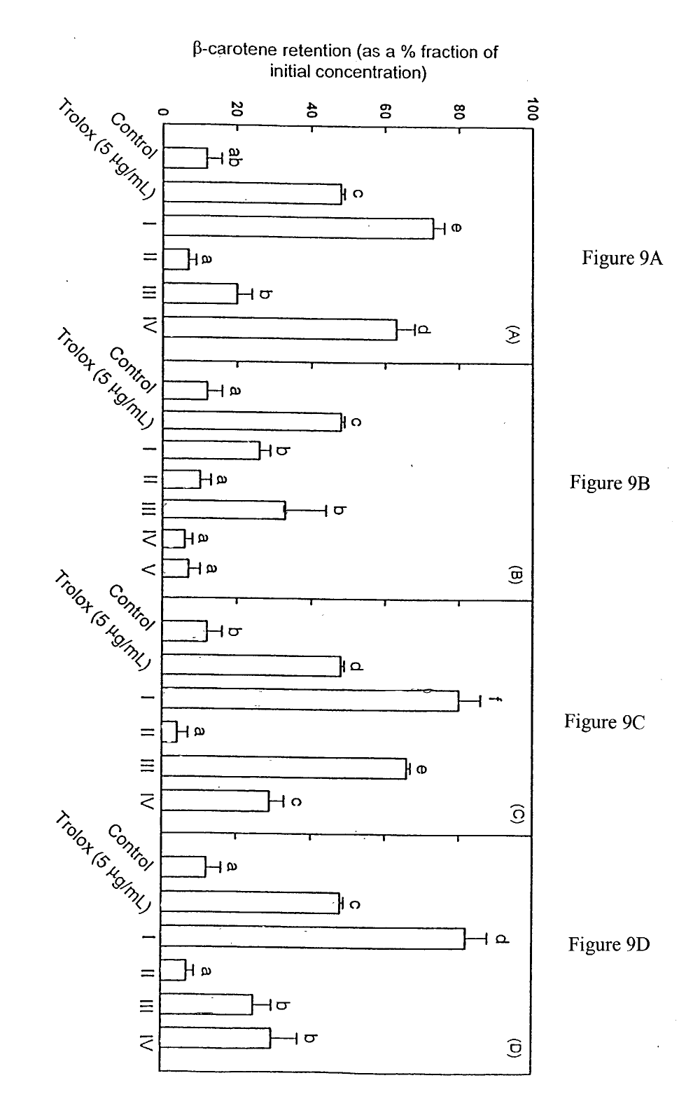

Assay media containing fractions I and IV of aqueous red beetroot extracts had ˜60-70% retention of β-carotene while that containing fractions III had <20% retention (FIG. 9A). β-Carotene retention in assay media, containing fractions I and III of ethanolic red beetroot extracts was <30% (FIG. 9B). Fractions I, III and IV of aqueous high-pigment beetroot extracts showed β-carotene retentions of about 80, 70 and 30%, respectively (FIG. 9C). Amongst the fractions of ethanolic high-pigment beetroots, only fraction I exerted a strong protecting effect towards β-carotene (FIG. 9D). Again, the antioxidant property seen in the extracts was also observed in the fractions, which showed different levels of β-carotene retention. Thus, the fractions retain various levels of antioxidant properties. The location of the antioxidant properties, as identified by ORAC, can be further identified.

### EXAMPLE 6

### INDUCER POTENCY

Example 6a: Assay of Inducer Potency. This assay used 1c1c7 murine hepatoma cells cultured in two 96-well microtiter plates as described by Prochaska and Santamaria (1988). One plate was used for the quinone reductase assay while the other was for the cell density measurement. Each plate containing 10,000 cells in MEM/well was incubated for 24 h, emptied and then 200 μL of serially diluted test materials (0-5 mg/mL) in MEM were added into wells. In each plate, there were two lanes of wells devoted to a no-cell blank and a cell control devoid of test materials. Wells in these two lanes contained MEM in place of test materials. After incubating for 48 h, the wells of one plate were emptied and the cells were lysed using 50 μL of 0.08% (w/v) aqueous digitonin solution (this solution was centrifuged to obtain a particle-free solution). The plate was then incubated for 20 min in a shaker oven at 37° C. and removed from the oven. A 150 μL aliquot of an aqueous assay reagent containing fetal bovine serum (0.066%, w/v), Tris-Cl (2.5%, v/v), Tween 20 (0.67%, v/v), FAD (0.67%, v/v), glucose-6-phosphate (0.1%, v/v), NADP (0.002%, w/v), glucose-6-phosphate dehydrogenase (0.0007%, w/v), MTT (0.03%, w/v), menadione (0.0008%, w/v) and acetonitrile (0.1%, v/v; used to prepare menadione solution) was added into each well. The absorbance of the reduced tetrazolium dye was measured over a 10-min period using an optical microtiter plate scanner (SPECTRA MAX plus, Molecular Devices, Sunnyvale, CA) set at 490 nm. The absorbance values of no-cell blanks were subtracted from those of the control and treated wells.

The second plate was emptied, kept immersed in a crystal violet bath for 10 min and rinsed under cold running water to remove excess stain. A 200 μL aliquot of a 0.5% (w/v) SDS solution (prepared in 50% aqueous ethanol) was added into each well and the plates were incubated for 1 h in a shaker oven set at 37° C. Plates were removed and the absorbance of the crystal violet was measured at 610 nm. The absorbance values of no-cell blanks were subtracted from those of the controls and treated. The degree of staining as reflected by the absorbance values of crystal violet was used as a measure of cell density.

For a given test material, the quinone reductase specific activity as induced by test compounds was calculated using both the absorbance value at 5 min in the quinone reductase assay and the absorbance value of the crystal violet assay. Ratio quinone reductase activity was the ratio between treated and control. This ratio was then converted to ng β-naphthoflavone (a known inducer) equivalents using a nonlinear equation obtained for an activity standard curve of β-naphthoflavone.

### Example 6b

Inducer Potency of Extracts.

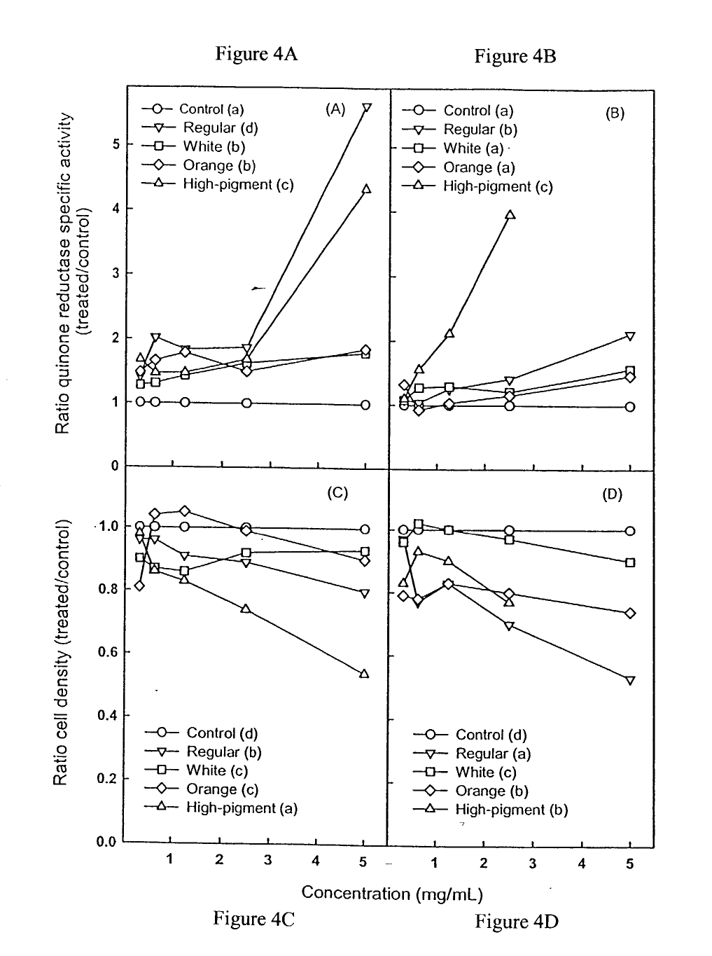

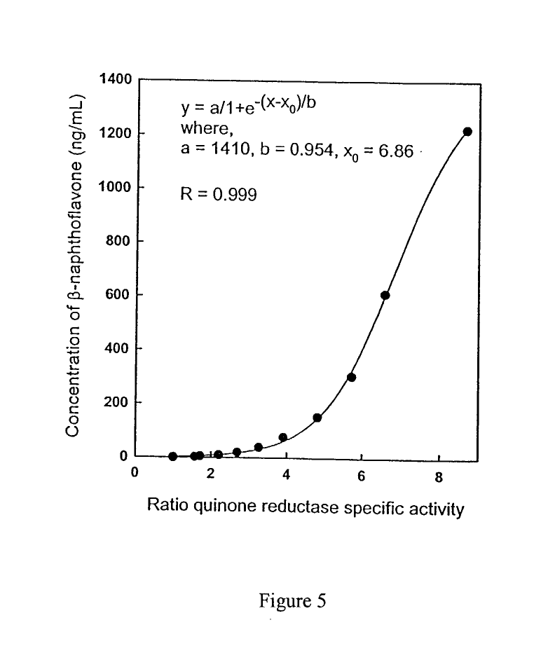

FIG. 4 shows the effects of both aqueous (4A and C) and ethanolic (4B and D) beetroot extracts on the induction of quinone reductase and cell density in Hepa 1c1c7 cells while Table 1 below shows the concentration (see FIG. 5 for standard curve and equation) of inducing agents in extracts. Aqueous extracts of beets at 5 mg/mL brought about a significant (p<0.05) elevation of quinone reductase activity, with the effects of both red and high-pigment beetroot extracts being superior to those of the other two varieties. As shown in FIG. 4B, the inducer potency of ethanolic extracts of red beetroots was much lower than that observed for its aqueous counterparts. Furthermore, the ethanolic extracts of high-pigment beetroots at 5 mg/mL caused a loss of cell viability (either toxicity or loss of adherent properties) of hepatoma cells in the bioassay (FIG. 4D). Nevertheless, it was evident that ethanolic extracts of red and high-pigment beetroots brought about a significant (p<0.05) increase in the quinone reductase activity while those of the other two varieties had no effect (p>0.05). The outcome of the quinone reductase assay is in good agreement with that of the other antioxidant assays. Therefore, red and high-pigment beetroot varieties exhibit the ability to induce Phase II enzymes and were chosen for further investigation as is described below.

TABLE 1
Concentration of phase II enzyme inducing components in red and high-
pigment beet extracts and their most active fractions^{a}

| | | ng β-naphthoflavone equivalents/mg | |
|---|---|---|---|
| test material | | aqueous | ethanolic |
| red | extract | 61.0 ± 12.0 | 1.94 ± 0.77 |
| | fraction I | 6.33 ± 0.44 | 4.62 ± 0.46 |
| | fraction II | — | 8.06 ± 1.37 |
| high-pigment | extract | 18.8 ± 5.26 | 12.8 ± 3.06 |
| | fraction I | 9.21 ± 1.75 | 11.2 ± 1.45 |
| | fraction II | ≦ | 8.95 ± 1.16 |
| | fraction IV | 28.3 ± 1.98 | 14.2 ± 2.55 |

^{a}Results are mean value of four replicates ± standard deviation and based upon the highest ratio quinone reductase specific activity observed for a given test material.

### Example 6c

Inducer Potency of Fractions.

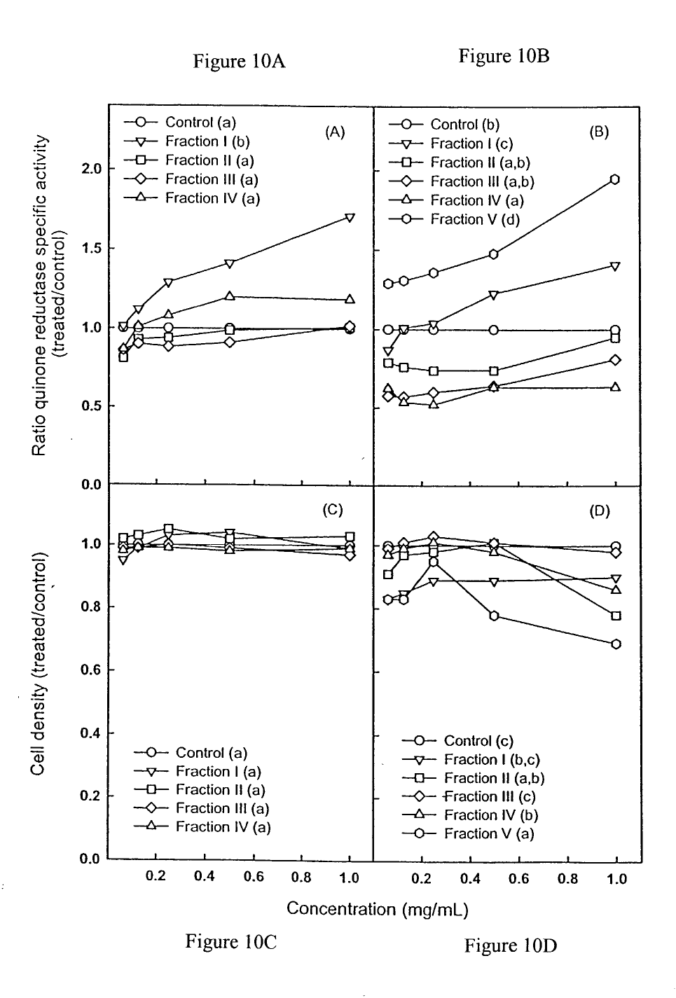

Amongst the fractions of aqueous extracts of red beetroots, fraction I showed the highest inducer potency (FIG. 10A) and its effect at 1 mg/mL was significantly (p<0.05) higher than that of the other fractions at the same concentration. Amongst the fractions of ethanolic extracts of red beetroots, fractions I and V exerted a high induction of quinone reductase activity while the other three fractions failed to show any significant effect (FIGS. 10B). Fractions of aqueous extracts of red beetroots caused lesser losses in cell viability than those of ethanolic extracts (FIG. 10C and 10D). Losses in cell viability of all fractions was less than 50% as indicated by ratio cell densities.

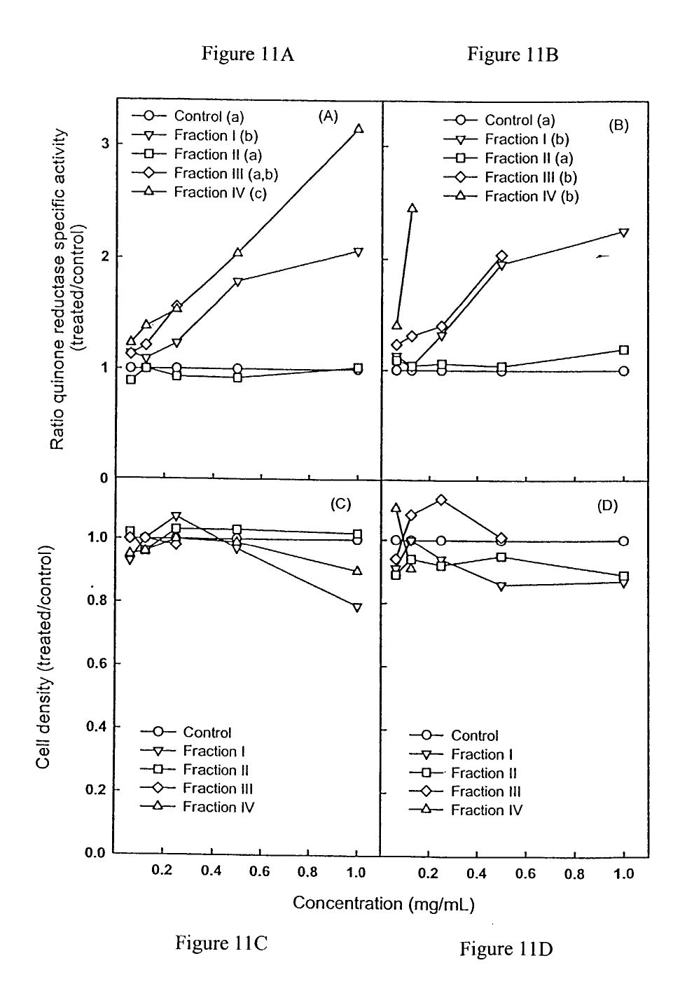

As shown in FIG. 11A, fraction IV of aqueous extracts of high-pigment beetroots at 1 mg/mL concentration exhibited the greatest induction of quinone reductase activity while fraction I was also increased the enzyme activity. Both fractions II and III were ineffective and the latter caused losses in cell viability at concentrations higher than 0.25 mg/mL (FIG. 11C). Induction of quinone reductase by fraction I of ethanolic extracts of high-pigment beetroots was comparable to that observed for fraction I of aqueous extracts (FIG. 11B), while fraction II had no inducing effect. Fractions III and IV increased quinone reductase activity at lower concentrations, but they caused losses in cell viability at higher concentrations (FIG. 11D). Thus, the ability to induce Phase II enzymes was present in several fractions of the extracts.

### EXAMPLE 7

### Further Isolation and Characterization of the Chromatography-Purified Fractions

### Example 7a

Thin-Layer Chromatography.

The most active column chromatographic fractions that were located as explained above in Example 2 were subjected to preparative silica gel thin-layer chromatography. Fraction I of aqueous and ethanolic extracts of both the red and high-pigment varieties was chromatographed using chloroform/methanol/water (55/85/20, v/v/v) as the mobile phase. The mobile phase for fraction V of ethanolic extracts of red beetroots contained chloroform/methanol/water/acetic acid (20/20/20/0.5, v/v/v/v), while that for fraction IV of aqueous high-pigment beetroots consisted of equal volumes of acetonitrile, ether and hexane.

### Example 7b

High-Performance Liquid Chromatography-Electrospray Ionization-Mass Spectrometry (HPLC-ESI-MS).

Positive electrospray mass spectra were recorded using a PE SCIEX API 365 LC/MS system (Applied Biosystems, Lincoln Center Drive, Foster City, CA) equipped with a C₁₈ column (4 μm layer thickness, 100×2 mm i.d., Phenomenex). Electrospray voltage, capillary temperature and sheath gas applied were 4.5 kV, 220° C. and N₂, respectively. A gradient elution system changing from 10% (v/v) of 0.2% (v/v) aqueous acetic acid in acetonitrile (B) and 90% (v/v) of 0.2% (v/v) aqueous acetic acid (A) to 50% (v/v) B in (A+B) was used. The flow rate was set at 70 μL/min for the first 10 min of elution. The elution was carried out under isocratic conditions during the final 10 min. The volume injected was 2 μL (Kobayashi et al., 2000).

### Example 7c

Electrospray Ionization-Mass Spectrometry/Mass Spectrometry (ESI-MS/MS).

A slightly modified method of Kujala et al. (2000) was employed. Sample in 50% (v/v) aqueous methanol was introduced using an ABI 140D solvent delivery system (Perkin-Elmer, Norwalk, CT) at a constant flow rate of 0.4 mL/h. The voltages applied were 4000, 41 and 210 for needle, orifice and ring, respectively. The collision gas energy was 25 V. Nebulizer, curtain and collision gas flow settings were 8, 12 and 3, respectively. Mass range was m/z 100 to 1000 with 0.2 amu step sizes.

### Example 7d

Thin-Layer Chromatography, HPLC-ESI-MS, and ESI-MS/MS of Fraction I of Aqueous and Ethanolic Extracts of Red and High-Pigment Beetroots.

Thin-layer chromatography of fraction I of aqueous red beetroot extracts yielded four bands at R_{f} 0.50 (pale yellow), 0.89 (orange), 0.92 (red) and 0.98 (yellow). LC/MS data revealed that the pale yellow band with R_{f} of 0.50 contained betalamic acid [(a), RT=20.3 min, λₘₐₓ=405 nm, (M−H⁺)⁻=219] while the orange band with R_{f} 0.89 contained unidentified betaxanthins. However, the MS/MS signals of the compounds were weak due to very low concentrations of the compounds. LC/MS data for the red band with R_{f} 0.92 showed the presence of four major betalains, the structures of which are shown below, namely vulgaxanthin I [(b), RT=5.1 min, λₘₐₓ=468 nm, M+H⁺=340], vulgaxanthin II [(c), RT=10.4 min, λₘₐₓ=469 nm, M+H⁺=341], betanidin [(d), RT=19.2 min, λₘₐₓ=540 nm, M+H⁺=389] and phyllocactin [(e), RT=25.4 min, λₘₐₓ=549 nm, M+H⁺=637]. MS/MS of M+H⁺ of these compounds, except phyllocactin, produced strong signals indicating the high concentration of betalains in the red band. The yellow band with R_{f} 0.98 consisted mainly of betanidin (d), but the MS/MS produced weak signals for this compound.

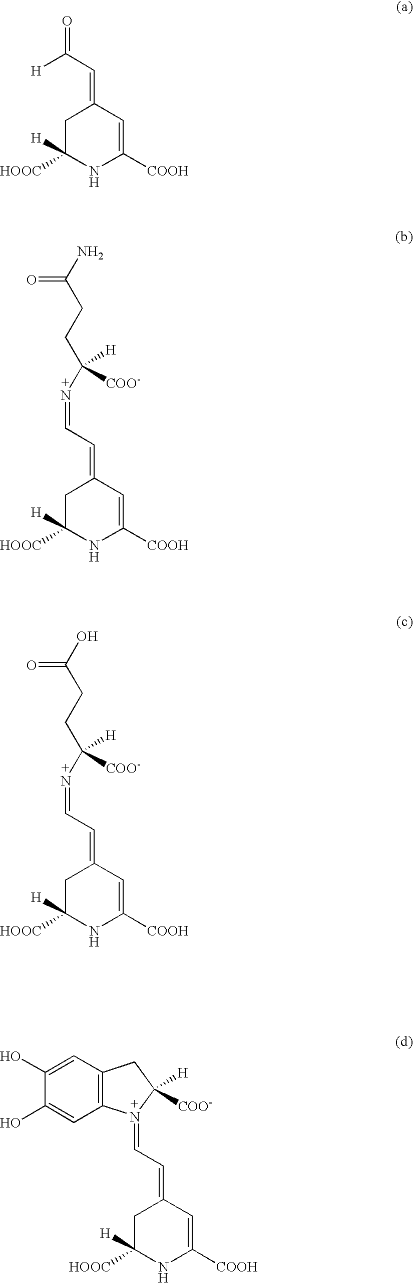

The pale yellow and orange color bands of fraction I of aqueous red beetroot extracts seen on TLC plates were absent in fraction I of aqueous high-pigment beetroot extracts. Two prominent bands were visible at R_{f} 0.92 (red) and 0.98 (yellow) and there was an orange color band (R_{f}=0.94) in between red and yellow bands. LC-MS and MS/MS data revealed that the red band isolated from fraction I of aqueous high-pigment beetroot extracts contained 2-descarboxybetanin [(f), RT=21.33 min, λₘₐₓ=532 nm, M+H⁺=507], betanin [(g), RT=21.93 min, λₘₐₓ=537 nm, M+H⁺=551] and 5″-O-E-feruloyl-2′-apiosyl-betanin [(h), RT=23.17 min, λₘₐₓ=548 nm, M+H⁺=859], the structures of which are shown below, in addition to all the betalains that identified in the red band isolated from fraction I of aqueous red beetroot extracts. The orange color band contained betalamic acid (a) and vulgaxanthin 11 (b) while the yellow spot contained decarboxylated betanidin (d).

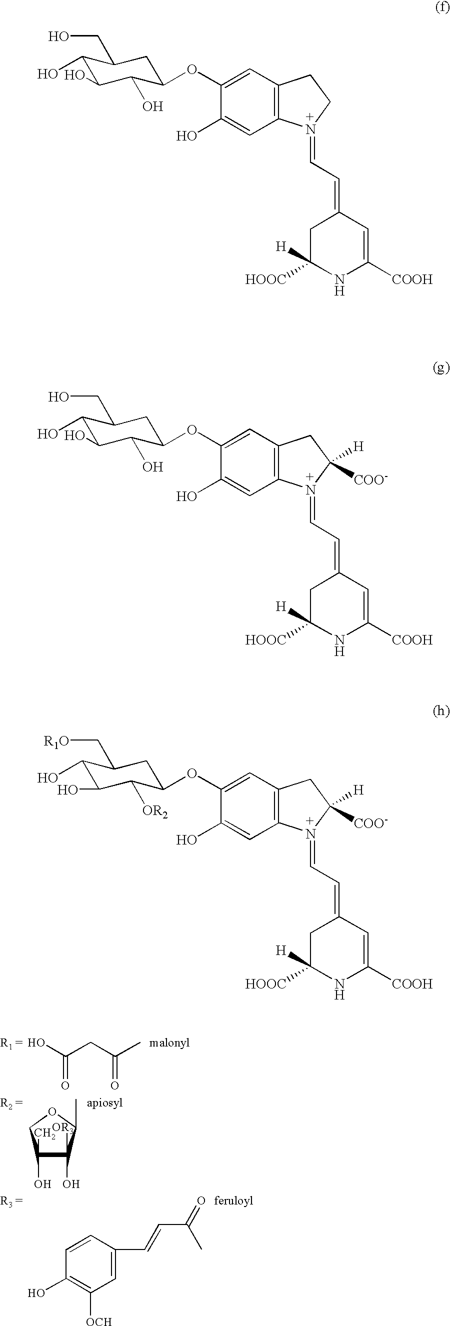

The several unidentified betacyanins present mainly in red TLC bands were suspected to be feruloyl esters of betanin because strong MS signals representative of a feruloyl moiety appeared at m/z 194, 195 and 196. As expected, LC-MS and MS/MS analyses of TLC bands isolated from fraction I of ethanolic extracts of both the varieties showed the occurrence of the same betalains profiles as in the aqueous extracts. Thus, fraction I of the aqueous and ethanolic extracts of red and high-pigment beetroots contains several different betalains, demonstrating that the betalains account for the antioxidant and Phase II enzyme inducing ability of the extracts.

### Example 8e

Thin-Layer Chromatography, HPLC-ESI-MS, and ESI-MS/MS of Fraction V of Ethanolic Extracts of Red Beetroots.

Separation of different classes of compounds present in the fraction V of ethanolic extracts of red beetroots on preparative TLC plates was poor and the recovered bands produced very weak signals when subjected to LC-MS. Therefore, this fraction, as such, was used for LC-MS and relatively strong signals were seen. LC/MS data showed the presence of two major compounds in this fraction, but only one compound was positively identified. The compound that produced M+H⁺ at m/z 713 was identified as betanidin-5-O-diglucoside (one more glucose residue attached to compound g), RT=19.2 min, 80 ₘₐₓ 537 nm). The unidentified compound produced M+H⁺ at m/z 962 (RT=23.12 min, λₘₐₓ 549 nm). A major fragment of this compound appeared at m/z 235. Several minor fragments were seen at m/z 178, 276, 353 and 478. Thus, fraction V of ethanolic extracts of red beetroots contains at least one betalain, further indicating that betalains account for the antioxidant and Phase II enzyme inducing ability of the extracts.

### Example 8f

Thin-Layer Chromatography, HPLC-ESI-MS, and ESI-MS/MS of Fraction IV of Aqueous and Ethanolic Extracts of Red Beetroots.

Similar to the compounds in fraction V of ethanolic extracts of red beetroots, the resolution of compounds in fraction IV of both the varieties on TLC plates was poor. Therefore, these fractions were subjected to LC/MS without carrying out further separation. LC/MS data revealed striking similarities between the fraction IV of red beetroots and that of the high-pigment beetroots. Both the varieties contained vulgaxanthin II [(c), RT=10.4 min, λₘₐₓ 469 nm, M+H⁺=341] and phyllocactin [(e), RT=25.4 min, λₘₐₓ 549 nm, M+H⁺=637] and their MS signal intensities were very strong as compared to relatively weak signals seen for them in the fraction I. Thus, fraction IV of aqueous and ethanolic extracts of red beetroots contains various betalains, further demonstrating that betalains account for the antioxidant and Phase II enzyme inducing ability of the extracts.

Statistical Analyses.

All experiments used completely randomized block designs (CRD) and the significance (p<0.05) of differences among treatment means was established using one-way analysis of variance (ANOVA) followed by the Tukey's studentized range test (Snedecor and Cochran, 1980).

Antioxidant and Phase II enzyme inducing activities of beetroots are variety-dependent and are predominantly associated with the red varieties. Lack of noticeable antioxidant and phase II enzyme inducing activities in white and orange beetroots coupled with contrastingly strong activities of the red beetroots indicate that the red beet pigments and their associated compounds may be responsible for the observed trends. Data for partially purified red and purple pigments of both the varieties further support this conclusion.

Fraction I of aqueous and ethanolic extracts of both the red beetroot varieties contains betalains that include betalamic acid, two betaxanthins and five betacyanins. Possession of malonyl and feruloyl moieties in two of the betacyanins indicates that these betacyanins may act as strong phase II enzyme inducers. Feruloylated betalains may also act as strong electron or hydrogen donors making them strong free radical scavengers. Although no esters of phenolic acids were detected in fraction V of ethanolic red beetroot extracts and fraction IV of aqueous and ethanolic extracts of both the red varieties, the presence of betalains as a major constituent in these fractions suggests their possible involvement in antioxidant and phase II enzyme-inducing activities. Furthermore, the possibility of metabolic products of betalains participating in cellular antioxidant and phase II enzyme inducing activities cannot be ruled out.

It is understood that the various preferred embodiments are shown and described above to illustrate different possible features of the invention and the varying ways in which these features may be combined. Apart from combining the different features of the above embodiments in varying ways, other modifications are also considered to be within the scope of the invention. The invention is not intended to be limited to the preferred embodiments described above.

### BIBLIOGRAPHY

Bresnick, E.; Birt, D. F.; Wolterman, K.; Wheeler, M.; Markin, R. S. Reduction in mammary tumorigenesis in the rat by cabbage and cabbage residue. Carcinogenesis 1990, 11, 1159-1163.

Bokern, M.; Heuer, S.; Wray, V.; Witte, L.; Macek, T.; Vanek, T.; Strack, D. Ferulic acid conjugates from cell cultures of Beta vulgaris. Phytochemistry 1991, 30, 3261-3265.

Burr, M. C.; Sweetnam, P. M. Vegetarianism, dietary fibre, and mortality. Am. J. Clin. Nutr. 1982, 36, 673-677.

Counts, J. L.; Goodman, J. I. Title. Mol. Carcinog. 1994, 11, 185-188.

Delgado-Vargas, F.; Jimenez, A. R.; Paredes-Lopez, O. Natural pigments: carotenoids, anthocyanins, and betalains-characteristics, biosynthesis, processing, and stability. Crit. Rev. Food Sci. Nutr. 2000, 40, 173-289.de Long, M. J.; Prochaska, H. J.; Talalay, P. Induction of NAD(P)H: quinone reductase in murine hepatoma cells by phenolic antioxidants, azo dyes, and other chemoprotectors: A model system for the study of anticarcinogens. Proc. Natl. Acad. Sci. U.S.A. 1986, 83, 787-791.

Escribano, J.; Pedreno, M. A.; Garcia-Carmona, F.; Munoz, R. Characterization of the antiradical activity of betalains from Beta vulgaris L. roots. Phytochem. Anal. 1998, 9, 124-127.

Goldman, I. L., A List of Germplasm Releases from the University of Wisconsin Table Beet Breeding Program, 1964-1992, HortScience 31(5):880-881 (1996).

Goldman, I. L., et al., Simultaneous Selection is Effective in Increasing Betalain Pigment Concentration but not Total Dissolved Solids in Red Beet, J. Amer. Soc. Hort. Sci. 121(1):23-26 (1996).

Guyonnet, D.; Siess, M.-H.; Le Bon, A.-M.; Suschetet, M. Modulation of phase II enzymes by organosulfur compounds from Allium vegetables in rat tissues. Toxicol. Applied Pharmacol. 1999, 154, 50-58.

Ioannides, C.; Parke, D. V. The cytochromes P-448. A unique family of enzymes involved in chemical toxicity and carcinogenesis. Biochem. Pharmacol. 1987, 36, 4197-4207.

Kapadia, G. J., et al. Chemoprevention of lung and skin cancer by Beta vulgaris (beet) root extract. Cancer Lett. 1996, 100:211-214.

Kobayashi, N.; Schmidt, J.; Nimtz, M.; Wray, V.; Schliemann, W. Betalains from Christmas cactus. Phytochemistry 2000, 54, 419-426.

Murakami A.; Ohigashi, H.; Koshimizu, K. Anti-tumor promotion with food phytochemicals: a strategy for cancer chemoprevention. Biosci. Biotech. Biochem. 1996, 60, 1-8.

Pellegrini, G.; Miller, N.; Rice-Evans, C. A. Screening of dietary carotenoids and carotenoid-rich fruit extracts for antioxidant activities applying 2,2′-Azino-bis(3-ethylbenzthiazoline-6-sulfonic acid). Methods in Enzymology 1999, 299, 379-389.

Prochaska, H. J.; Santamaria, A. B. Direct measurement of NAD(P)H:Quinone reductase from cells cultured in microtiter wells: a screening assay for anticarcinogenic enzyme inducers. Analytical Biochemistry 1988, 169, 328-336.

Prochaska, H. J.; Santamaria, A. B.; Talalay, P. Rapid detection of inducers of enzymes that protect against carcinogens. Proc. National Acad. Sci. U.S.A. 1992, 89, 2394-2398.

Prochaska, et al., Rapid detection of inducers of enzymes that protect against carcinogens, Proc. Natl. Acad. Sci. USA 89: 2394-2398 (1996).

Talalay, P. Mechanisms of induction of enzymes that protect against chemical carcinogenesis. Adv. Enzyme Regul. 1989, 28, 149-159.

Tawfiq, N.; Heaney, R. K.; Plumb, J. A.; Fenwick, C. R.; Musk, S. R. R.; Williamson, G. Dietary glucosinolates as blocking agents against carcinogenesis: glucosinolate breakdown products assessed by induction of quinone reductase activity in murine hepa1c1c7 cells. Carcinogenesis 1995, 16, 1191-1194.

Velioglu, Y. S.; Mazza, G.; Gao, L.; Oomah, B. D. Antioxidant activity and total phenolics in selected fruits, vegetables, and grain products. J. Agric. Food Chem. 1998, 46, 4113-4117.

Vereltzis, K. P.; Buck, E. M. Color stability and sensory attributes of chicken frankfurters made with betalains and potassium sorbate versus sodium nitrite. J. Food Protect. 1984, 47, 41-45.

Vereltzis, K.; Buck, E. M.; Labbe, R. G. Effectiveness of a betalains/potassium sorbate system versus sodium nitrite for color development and control of total aerobes, Clostridium perfringens and Clostridium sporogenes in chicken frankfurters. J. Food Protect. 1984, 47, 532-536.

von Elbe, J. H.; Klement, J. T.; Amundson, C. H.; Cassens, R. G.; Lindsay, R. C. Evaluation of betalain pigments as sausage colorants. J. Food Sci. 1974, 39, 128-132.

Wattenberg, L. W. Inhibition of neoplasia by minor dietary constituents. Cancer Res. 1983, 43, 2448s-2453s.

Wattenberg, L. W. Chemoprevention of cancer. Preventive Med. 1996, 25, 44-45.

Wilkinson, J.; Clapper, M. L. Detoxification enzymes and chemoprevention. Proc. Soc. Experimental Biol. Med. 1997, 216, 192-200.

Willett, C. W. Micronutrients and cancer risk. Am. J. Clin. Nutr. 1994, 59, 162S-165S.

Wolyn, D. J. & Gabelman, W. H., Inheritance of Root and Petiole Pigmentation in Red Table Beet, J of Heredity, 80(1):33-38 (1989).

Zakharova, N. S.; Petrova, T. A. Investigation of betalains and betalain oxidase of leaf beet. Appl. Biochem. Microbiol. 1997, 33, 481-484.

What is claimed is:

1. A method of increasing the chemoprotective amount of at least one Phase II enzyme in a mammal, the method comprising administering an effective quantity of an extract comprising a betalain.

2. A method of claim 1, wherein the Phase II enzyme that is increased comprises quinone reductase.

3. A method of claim 1, wherein the betalain is administered as an extract of the betalain.

4. A method of claim 3, wherein the betalain is administered as an aqueous extract of the betalain.

5. A method of claim 3, wherein the betalain is administered as an aqueous ethanol extract of the betalain.

6. A method of claim 1, wherein the betalain is administered as a fraction that has been isolated by chromatography.

7. A method of extracting a betalain from a source of the betalain, the method comprising:
(a) freeze-drying the source containing the betalain;
(b) grinding the freeze-dried source; and
(c) extracting the betalain from the ground source with a solvent.

8. A method of claim 7, wherein the betalain is extracted with a water-containing solvent.

9. A method of claim 8, wherein the betalain is extracted with water.

10. A method of claim 9, wherein the betalain is extracted with water at about 100° C.

11. A method of claim 8, wherein the betalain is extracted with aqueous ethanol.

12. A method of claim 11, wherein the betalain is extracted with aqueous ethanol at 5% water and 95% ethanol (volume/volume).

13. A method of claim 7, wherein the betalain is extracted from a plant.

14. A method of claim 8, wherein the betalain is extracted from a fungus.

15. A method of claim 7, further comprising isolating the betalain extract into components.

16. A method of claim 15, wherein chromatography is used to isolate the betalain components.

17. A nutritional supplement comprising at least 0.3% of a betalain.

18. A nutritional supplement of claim 17, wherein the nutritional supplement comprises at least about 0.4% of the betalain.

19. A nutritional supplement of claim 18, wherein the nutritional supplement comprises at least about 0.8% of the betalain.

20. A nutritional supplement comprising a betalain prepared in accordance with claim 7.
